(19) United States
(12) Patent Application Publication
(10) Pub. No.: US 2004/0121385 A1
(43) Pub. Date: Jun. 24, 2004

(54) Variants of the human AMP-activated protein kinase gamma 3 subunit

(75) Inventors: Leif Andersson, Uppsala (SE); L. Holger Luthman, Bromma (SE); Stefan Marklund, Uppsala (SE)

Correspondence Address: FISH & RICHARDSON P.C., 3300 DAIN RAUSCHER PLAZA, 60 SOUTH SIXTH STREET, MINNEAPOLIS, MN, 55402, US

(73) Assignee: Arexis AB, a Swedish corporation

(21) Appl. No.: 10/705,137

(22) Filed: Nov. 10, 2003

Related U.S. Application Data
(63) Continuation of application No. 09/826,581, filed on Apr. 5, 2001, now abandoned.
(60) Provisional application No. 60/195,665, filed on Apr. 7, 2000.

(51) Int. Cl. C12Q 1/68; C07H 21/04; C07K 14/705

(52) U.S. Cl. 435/6; 435/69.1; 435/320.1; 435/325; 530/350; 536/23.5

(57) ABSTRACT

PRKAG3 nucleotide and amino acid sequence variants and methods of detecting such sequence variants are described. Methods for providing risk estimates for development of a metabolic disease also are described and are based on the presence or absence of PRKAG3 sequence variants in a biological sample.

### TECHNICAL FIELD

This invention relates to new variants of the γ3 subunit of human AMP-activated protein kinase (PRKAG3), to genes encoding the variants, and uses thereof.

### BACKGROUND

AMP-activated protein kinase (AMPK) has a key role in regulating the energy metabolism in the eukaryotic cell. See, for example, Hardie et al., Annu. Rev. Biochem., 67:821-855,1998; Kemp et al., TIBS, 24:22-2.5, 1999. Mammalian AMPK is a heterotrimeric complex comprising a catalytic α subunit and two non-catalytic β and γ subunits that regulate the activity of the α subunit. The yeast homologue (denoted SNF1) of this enzyme complex has been well characterized; it comprises a catalytic chain (Snf1) corresponding to the mammalian α subunit, and regulatory subunits: Sip1, Sip2 and Gal83 corresponding to the mammalian β subunit, and Snf4 corresponding to the mammalian γ subunit. Sequence data show that AMPK homologues also exist in Caenorhabditis elegans and Drosophila.

It has been observed that mutations in yeast SNF1 and SNF4 cause defects in the transcription of glucose-repressed genes, sporulation, thermotolerance, peroxisome biogenesis, and glycogen storage.

In mammalian cells, AMPK has been proposed to act as a “fuel gauge.” It is activated by an increase in the AMP:ATP ratio, resulting from cellular stresses such as heat shock and depletion of glucose and ATP. Activated AMPK turns on ATP-producing pathways (e.g. fatty acid oxidation) and inhibits ATP-consuming pathways (e.g., fatty acid and cholesterol synthesis), through phosphorylation of the enzymes acetyl-CoA carboxylase and hydroxymethylglutaryl-CoA (HMG-CoA) reductase. It has also been reported to inactivate in vitro glycogen synthase, the key regulatory enzyme of glycogen synthesis, by phosphorylation (Hardie et al., 1998, supra); whether glycogen synthase is a physiological target of AMFK in vivo remains unclear, however.

Several isoforms of the three different AMPK subunits are present in mammals. An RN allele in Hampshire pigs is associated with a non-conservative mutation in a gene encoding a muscle-specific isoform of the AMPK γ chain. In humans, PRKAAl on human chromosome (HSA) 5pl2 and PRKAA2 on HSAlp31 respectively encode isoforms α1 and α2 of the α subunit, PRKABl on HSAl2q241, and PRKAB2 (not yet mapped) respectively encode isoforms β1 and β2 of the β subunit, and PRKAGl on HSA12q13.1 and PRKAG2 on HSA7q35-q36 respectively encode isoforms γ1 and γ2 of the γ subunit (OMIM database, http://www.ncbi.nlm.nih.gov/omim/, July 1999). A third isoform (γ3) of the γ subunit of AMPK also is present. Milan et al., Science, 2000, in press; and Cheung et al., Biochem. J., 2000, 346:659-669. Analysis of the sequences of these γ subunits shows that they include four cystathione β synthase (CBS) domains whose function is unknown.

### SUMMARY

The invention is based on the identification of nucleotide and amino acid sequence variants in the human PRKAG3 gene. The sequence variants may be associated with metabolic diseases such as diabetes and obesity, leading to genetic tests that can increase the accuracy in diagnosis and treatment of such diseases in humans.

In one aspect, the invention features an isolated nucleic acid including a human PRKA3 sequence, wherein the PRK4G3 sequence includes a nucleotide sequence variant and nucleotides flanking the sequence variant, and wherein the isolated nucleic acid is at least 15 base pairs in length. The nucleotide sequence variant can be associated with a metabolic disease such as diabetes or obesity. The nucleotide sequence variant can be in an exon, e.g. exon 3, exon 4, or exon 10. An exon 3 variant can include a substitution of a guanine for a cytosine at nucleotide 320; an exon 4 variant can include a substitution of a thymine for a cytosine at nucleotide 550; and an exon 10 variant can include a substitution of a thymine for a cytosine at nucleotide 1037. A nucleotide sequence variant also can be in an intron such as intron 6. The PRKAG3 nucleic acid sequence can encode an AMP-activated protein kinase γ3 subunit polypeptide that includes an amino acid sequence variant. The amino acid sequence variant can include substitution of an alanine residue for a proline residue at amino acid 71 or substitution of a tryptophan residue for an arginine residue at amino acid 340.

The invention also features a method for determining a risk estimate of a metabolic disease in a subject. The method includes detecting the presence or absence of a PRKAG3 nucleotide sequence variant in the subject, and determining the risk estimate based, at least in part, on presence or absence of the variant in the subject. Metabolic diseases include, for example, diabetes and obesity.

In another aspect, the invention features a method for detecting a PRKAG3 polypeptide variant in a subject. The method includes providing a biological sample from the subject, contacting the biological sample with an antibody having specific binding affinity for the PRKAG3 polypeptide variant, and detecting the presence or absence of the PRKAG3 polypeptide variant in the biological sample.

In yet another aspect, the invention features an article of manufacture that includes a substrate and an array of different nucleic acids immobilized on the substrate, wherein at least one of the different nucleic acids is a PRKAG3 nucleic acid, and wherein the PRKAG3 nucleic acid includes a PRKAG3 nucleotide sequence variant and nucleotides flanking the sequence variant. The array can include multiple PRKAG3 nucleic acids, wherein each of the PRKAG3 nucleic acids includes a different PRKAG3 nucleotide sequence variant and nucleotides flanking the variant.

Unless otherwise defined, all technical and scientific terms used herein have the same meaning as commonly understood by one of ordinary skill in the art to which this invention belongs. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples ate illustrative only and not intended to be limiting.

Other features and advantages of the invention will be apparent from the following detailed description, and from the claims.

### DESCRIPTION OF DRAWINGS

FIG. 1 is an 821 bp DNA sequence of PRKAG3 from the 5′ untranscribed and untranslated region (UTR) through intron 2, including exon 1 and 2.

FIG. 2 is a 989 bp DNA sequence of PRKAG3 from intron 2 through intron 4, including exons 3 and 4.

FIG. 3 is a 1722 bp DNA sequence of PRKAG3 from intron 4 through intron 10, including exons 5-10.

FIG. 4 is a 1014 bp DNA sequence of PRKAG3 from intron 10 through the 3-UTR, including exons 11-13.

FIG. 5 is the complete coding sequence of PRKAG3 (nucleotides 20-1489) and the amino acid sequence of the PRKAG3 polypeptide.

### DETAILED DESCRIPTION

The various aspects of the present invention are based upon the discovery and characterization of nucleotide and amino acid sequence variants of the human PRKAG3 gene.

Nucleotide Sequence Variants

As used herein, “nucleotide sequence variant” refers to any alteration in the wild-type gene sequence, and includes variations that occur in coding and non-coding regions, including exons, introns, promoters, and untranslated regions. In some instances, the nucleotide sequence variant results in a PRKAG3 polypeptide having an altered amino acid sequence. The term “polypeptide” refers to a chain of at least four amino acid residues. Corresponding PRKAG3 polypeptides, irrespective of length, that differ in amino acid sequence are herein referred to as allozymes. Certain PRKAG3 nucleotide variants do not alter the amino acid sequence. Such variants, however, could alter regulation of transcription as well as mRNA stability. Nucleotide variants also may be linked to functionally important mutations.

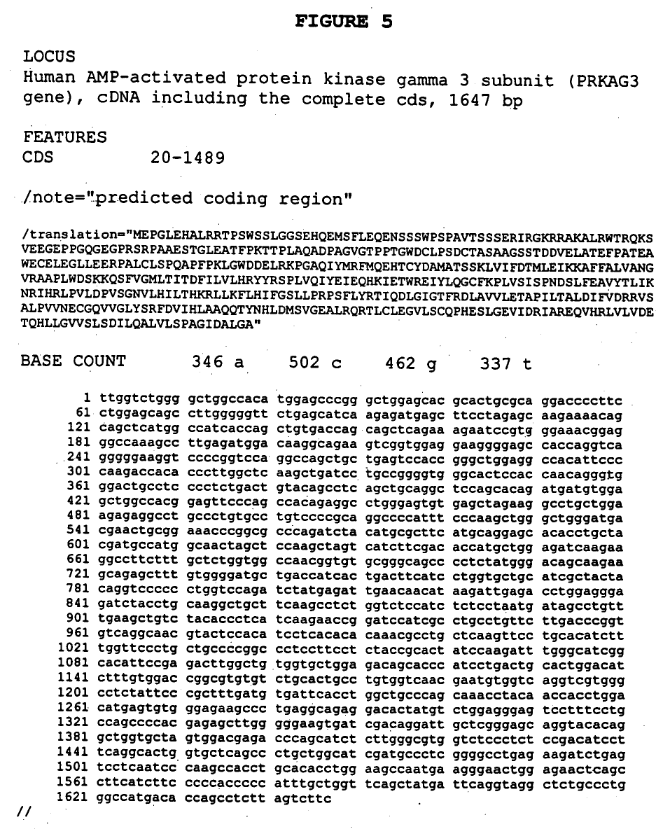

For example, the variant can be in exons 1-10, and in particular, in exon 3, 4, or 10. Numbering of variants within exons is according to the cDNA sequence of FIG. 5. An exon 3 variant can include, for example, a substitution of a guanine for a cytosine at nucleotide 230 (C230G). This substitution results in the substitution of an alanine residue for a proline residue at amino acid 71 (P71A). An exon 4 variant can include, for example, a thymine for a cytosine at nucleotide 559 (T559C). This does not result in an amino, acid change. An exon 10 variant can include, for example, substitution of a thymine for a cytosine at nucleotide 1037 (C1037T), resulting in the substitution of a tryptophan for an arginine residue at amino acid 340 (R340W).

Isolated nucleic acid molecules of the invention can be produced by standard techniques. As used herein, “isolated nucleic acid” refers to a sequence corresponding to part or all of a gene encoding human PRKAG3, but free of sequences that normally flank one or both sides of the gene in a mammalian genome. An isolated nucleic acid can be, for example, a DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule (e.g., a cDNA or genomic DNA fragment produced by PCR or restriction endonuclease treatment) independent of other sequences as well as recombinant DNA that is incorporated into a vector, an autonomously replicating plasmid, a virus (e.g., a retrovirus, adenovirus, or herpes virus), or into the genomic DNA of a prokaryote or eukaryote. In addition, an isolated nucleic acid can include a recombinant DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not to be considered an isolated nucleic acid.

Isolated nucleic acid molecules are at least about 15 base pairs in length. For example, the nucleic acid molecule can be about 15-25, 20-30, 22-32, 25-35, 40-50, 50-100, or greater than 150 base pairs in length, e.g., 200-300, 300-500, or 500-1000 base pairs in length. Such fragments, whether protein-encoding or not, can be used as probes, primers, and diagnostic reagents. In some embodiments, the isolated nucleic acid molecules encode a full-length PRKAG3 polypeptide. Nucleic acid molecules of the invention can be DNA or RNA, linear or circular, and in sense or antisense orientation.

Specific point changes can be introduced into the nucleic acid sequence encoding wild-type human PRKAG3 by, for example, oligonucleotide-directed mutagenesis. In this method, a desired change is incorporated into an oligonucleotide, which then is hybridized to the wild-type nucleic acid. The oligonucleotide is extended with a DNA polymerase, creating a heteroduplex that contains a mismatch at the introduced point change, and a single-stranded nick at the 5′ end, which is sealed by a DNA ligase. The mismatch is repaired upon transformation of E. coli or other appropriate organism, and the gene encoding the modified human PRKAG3 can be re-isolated from E. coli or other appropriate organism. Kits for introducing site-directed mutations can be purchased commercially. For example, Muta-Gene™ in-vitro mutagenesis kits can be purchased from Bio-Rad Laboratories, Inc. (Hercules, Calif.).

Polymerase chain reaction (PCR) techniques also can be used to introduce mutations. See, for example, Vallette et al., Nucleic Acids Res., 1989, 17(2):723-733. Polymerase chain reaction (PCR) techniques can be used to produce nucleic acid molecules of the invention. PCR refers to a procedure or technique in which target nucleic acids are amplified. Sequence information from the ends of the region of interest or beyond typically is employed to design oligonucleotide primers that are identical in sequence to opposite strands of the template to be amplified. For introduction of mutations, oligonucleotides that incorporate the desired change are used to amplify the nucleic acid sequence of interest. PCR can be used to amplify specific sequences from DNA as well as RNA, including sequences from total genomic DNA or total cellular RNA. Primers are typically 14 to 40 nucleotides in length, but can range from 10 nucleotides to hundreds of nucleotides in length. General PCR techniques are described, for example in PCR Primer: A Laboratory Manual, Ed. by Dieffenbach, C. and Dveksler, G., Cold Spring Harbor Laboratory Press, 1995.

Nucleic acids containing sequence variants also can be produced by chemical synthesis, either as a single nucleic acid molecule or as a series of oligonucleotides. For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a double-stranded nucleic acid molecule per oligonucleotide pair, which then can be ligated into a vector.

Detection of Sequence Variants

Human PRKAG3 nucleotide sequence variants described herein can be associated with a metabolic disease, such as diabetes or obesity. Risk estimates can be determined for a subject by determining if a particular sequence variant is present or absent in the subject. As used herein, “risk estimate” refers to the relative risk a subject has for developing a metabolic disease. For example, a risk estimate for development of diabetes can be determined based on the presence or absence of PRKAG3 variants. A subject containing, for example, the R340W PRKAG3 variant may have a greater likelihood of developing diabetes. Additional risk factors include, for example, family history of diabetes, obesity, sedentary life style, and other genetic factors. Detection of PRKAG3 sequence variants also can help in choosing the appropriate agent for treatment of the metabolic disease.

Nucleotide sequence variants can be assessed, for example, by sequencing exons and introns of the PRKAG3 gene, by performing allele-specific hybridization, allele-specific restriction digests, mutation specific polymerase chain reactions (MSPCR), oligonucleotide ligation assays, or by single-stranded conformational polymorphism (SSCP) detection. Reporter molecules used in assays for detecting sequence variants can include, for example, radioisotopes, fluorophores, and molecular beacons.

Genomic DNA is generally used in the analysis of PRKAG3 nucleotide sequence variants. Genomic DNA is typically extracted from peripheral blood samples, but can be extracted from such tissues as mucosal scrapings of the lining of the mouth or from renal or hepatic tissue. Routine methods can be used to extract genomic DNA from a blood or tissue sample, including, for example, phenol extraction, or proteinase K treatment of lysed cells, salt precipatation of proteins, and ethanol purification. Alternatively, genomic DNA can be extracted with kits such as the QIAamp® Tissue Kit (Qiagen, Chatsworth, Calif.), Wizards Genomic DNA purification kit (Promega, Madison, Wis.) and the A.S.A.P.™ Genomic DNA isolation kit (Boehringer Mannheim, Indianapolis, Ind.).

For example, exons and introns of the PRAKG3 gene can be amplified through PCR and then directly sequenced. This method can be varied, including using dye primer sequencing to increase the accuracy of detecting heterozygous samples. Alternatively, a nucleic acid molecule can be selectively hybridized to the PCR product to detect a gene variant. Hybridization conditions are selected such that the nucleic acid molecule can specifically bind the sequence of interest, e.g., the variant nucleic acid sequence. Such hybridizations typically are performed under high stringency as some sequence variants include only a single nucleotide difference. High stringency conditions can include the use of low ionic strength solutions and high temperatures for washing. For example, nucleic acid molecules can be hybridized at 42° C. in 2×SSC (0.3M NaCl/0.03M sodium citrate)/0.1% sodium dodecyl sulfate (SDS) and washed in 0.1×SSC (0.015M NaCl/0.0015M sodium citrate), 0.1% SDS at 65° C. Hybridization conditions can be adjusted to account for unique features of the nucleic acid molecule, including length and sequence composition.

Allele-specific restriction digests can be performed in the following manner. If a nucleotide sequence variant introduces a restriction site, restriction digest with the particular restriction enzyme can differentiate the alleles. For example, the C1037T change described herein results in the introduction of an MspI restriction site. Thus, the MspI restriction pattern can be assessed to determine if an allele contains the C1037T variant. Typically, PCR is performed to amplify a region of the PRKAG3 gene surrounding the variant prior to digestion with the restriction enzyme. For PRKAG3 variants that do not alter a common restriction site, primers can be designed that introduce a restriction site when the variant allele is present, or when the wild-type allele is present, or an oligonucleotide ligation assay can be used to detect such polymorphisms. See, Landegren et al., Science, 241:1077 (1988). For example, the C230G change results in an amino acid substitution (P71A), but does not alter a restriction site. In general, a PCR product that includes the mutant site is incubated with two oligonucleotides that hybridize side by side and that are positioned such that the 3′ end of one oligonucleotide is located at the polymorphic site. The oligonucleotides are ligated by DNA ligase if the nucleotides at the junction are correctly base-paired. The test can be carried out as separate reactions for the two alleles if a single reporter molecule is used, or in a single reaction if different reporter molecules are used.

Certain variants, such as insertion or deletion of one or more nucleotides, change the size of the DNA fragment encompassing the variant. The insertion of nucleotides can be assessed by amplifying the region encompassing the variant and determining the size of the amplified products in comparison with size standards. For example, the region containing the insertion or deletion can be amplified using a primer set from either side of the variant. One of the primers is typically labeled, for example, with a fluorescent moiety, to facilitate sizing. The amplified products can be electrophoresed through acrylamide gels using a set of size standards that are labeled with a fluorescent moiety that differs from the primer.

PCR conditions and primers can be developed that amplify a product only when the variant allele is present or only when the wild-type allele is present (MSPCR or allele-specific PCR). For example, patient DNA and a control can be amplified separately using either a wild-type primer or a primer specific for the variant allele. Each set of reactions is then examined for the presence of amplification products using standard methods to visualize the DNA. For example, the reactions can be electrophoresed through an agarose gel and DNA visualized by staining with ethidium bromide or other DNA intercalating dye. In DNA samples from heterozygous patients, reaction products would be detected in each reaction. Patient samples containing solely the wild-type allele would have amplification products only in the reaction using the wild-type primer. Similarly, patient samples containing solely the variant allele would have amplification products only in the reaction using the variant primer.

Mismatch cleavage methods also can be used to detect differing sequences by PCR amplification, followed by hybridization with the wild-type sequence and cleavage at points of mismatch. Chemical reagents, such as carbodiimide or hydroxylamine and osmium tetroxide can be used to modify mismatched nucleotides to facilitate cleavage.

Alternatively, PRKAG3 amino acid sequence variants can be detected by various immunoassays using antibodies having specific binding affinity for variant PRKAG3 polypeptides. Appropriate immunoassay methods are known in the art, including, for example, enzyme-linked immunosorbent assays (ELISA), radioimmunoassays (RIA), and fluorescence activated cell sorting (FACS).

Variant PRKAG3 polypeptides also can be detected by monitoring PRKAG3 kinase activity. Assays that monitor phosphorylation of PRKAG3 substrates, such as acetyl-CoA carboxylase or HMG-CoA reductase, can be performed using standard technology. In general, cellular extracts containing PRKAG3 polypeptides are incubated in a kinase buffer containing phosphate and an appropriate substrate, and phosphorylation of the substrate is monitored. For example, AMPK activity in muscle extracts can be assayed using ³²p labelled ATP and the SAMS peptide, as described by Davies et al., Eur. J. Biochem., 186:123-128 (1989).

Production of Antibodies

Antibodies having specific binding affinity for variant PRKAG3 polypeptides can be produced using standard methodology. Variant PRKAG3 polypeptides can be produced in various ways, including recombinantly. The cDNA nucleic acid sequence of PRKAG3 is provided in FIG. 5, (See GenBank Accession No. AF214520). Amino acid changes can be introduced by standard techniques, as described above.

A nucleic acid sequence encoding a PRKAG3 variant polypeptide can be ligated into an expression vector and used to transform a bacterial or eukaryotic host cell. In general, nucleic acid constructs include a regulatory sequence operably linked to a PRKAG3 nucleic acid sequence. Regulatory sequences do not typically encode a gene product, but instead affect the expression of the nucleic acid sequence. In bacterial systems, a strain of E. coli such as BL-21 can be used. Suitable E. coli vectors include the pGEX series of vectors that produce fusion proteins with glutathione S-transferase (GST). Transformed E. coli are typically grown exponentially then stimulated with isopropylthiogalactopyranoside (IPTG) prior to harvesting. In general, such fusion proteins are soluble and can be purified easily from lysed cells by adsorption to glutathione-agarose beads followed by elution in the presence of free glutathione. The pGEX vectors are designed to include thrombin or factor Xa protease cleavage sites so that the cloned target gene product can be released from the GST moiety.

In eukaryotic host cells, a number of viral-based expression systems can be utilized to express PRKAG3 variant polypeptides. A nucleic acid encoding a PRKAG3 variant polypeptide can be cloned into, for example, a baculoviral vector and then used to transfect insect cells. Alternatively, the nucleic acid encoding a PRKAG3 variant can be introduced into a SV40, retroviral or vaccinia based viral vector and used to infect host cells.

Mammalian cell lines that stably express PRKAG3 variant polypeptides can be produced by using expression vectors with the appropriate control elements and a selectable marker. For example, the eukaryotic expression vector pCR3.1 (Invitrogen, San Diego, Calif.) is suitable for expression of PRKAG3 variant polypeptides in, for example, COS cells. Following introduction of the expression vector by electroporation, DEAE dextran, or other suitable method, stable cell lines are selected. Alternatively, amplified sequences can be ligated into a mammalian expression vector such as pcDNA3 (Invitrogen, San Diego, Calif.) and then transcribed and translated in vitro using wheat germ extract or rabbit reticulocyte lysate. PRKAG3 variant polypeptides can be purified by standard protein purification techniques. As used herein, a “purified” PRKAG3 polypeptide has been separated from cellular components that naturally accompany it. Typically, the PRKAG3 polypeptide is purified when it is at least 60% (e.g., 70%, 80%, 90%, or 95%), by weight, free from proteins and naturally-occurring organic molecules that are naturally associated with it.

Various host animals can be immunized by injection of a purified, PRKAG3 variant polypeptide. Host animals include rabbits, chickens, mice, guinea pigs and rats. Various adjuvants that can be used to increase the immunological response depend on the host species and include Freund's adjuvant (complete and incomplete), mineral gels such as aluminum hydroxide, surface active substances such as lysolecithin, pluronic polyols, polyanions, peptides, oil emulsions, keyhole limpet hemocyanin and dinitrophenol. Polyclonal antibodies are heterogenous populations of antibody molecules that are contained in the sera of the immunized animals. Monoclonal antibodies, which are homogeneous populations of antibodies to a particular antigen, can be prepared using a PRKAG3 variant polypeptide and standard hybridoma technology. In particular, monoclonal antibodies can be obtained by any technique that provides for the production of antibody molecules by continuous cell lines in culture such as described by Kohler, G. et al., Nature, 256:495 (1975), the human B-cell hybridoma technique (Kosbor et al., Immunology Today, 4:72 (1983); Cole et al., Proc. Natl. Acad. Sci USA, 80:2026 (1983)), and the EBV-hybridoma technique (Cole et al., “Monoclonal Antibodies and Cancer Therapy”, Alan R. Liss, Inc., pp. 77-96 (1983). Such antibodies can be of any immunoglobulin class including IgG, IgM, IgE, IgA, IgD and any subclass thereof. The hybridoma producing the monoclonal antibodies of the invention can be cultivated in vitro and in vivo.

Antibody fragments that have specific binding affinity for a PRKAG3 variant polypeptide can be generated by known techniques. For example, such fragments include but are not limited to F(ab′)2 fragments that can be produced by pepsin digestion of the antibody molecule, and Fab fragments that can be generated by reducing the disulfide bridges of F(ab′)2 fragments. Alternatively, Fab expression libraries can be constructed. See, for example, Huse et al., Science, 246:1275 (1989). Once produced, antibodies or fragments thereof are tested for recognition of PRKAG3 variant polypeptides by standard immunoassay methods including ELISA techniques, RIAs, and Western blotting. See, Short Protocols in Molecular Biology, Chapter 11, Green Publishing Associates and John Wiley & Sons, Edited by Ausubel, F.M et al., 1992.

Nucleic Acid Arrays

The invention also features an article of manufacture that includes a substrate and an array of different nucleic acid molecules immobilized on the substrate. At least one of the different nucleic acid molecules includes a PRKAG3 nucleic acid. In some embodiments, the array of different nucleic acid molecules includes different PRKAG3 nucleic acid molecules, wherein each PRKAG3 nucleic acid includes a different PRKAG3 nucleotide sequence variant and nucleotides flanking the sequence variant. Such articles of manufacture allow complete haplotypes of patients to be assessed.

Suitable substrates for the article of manufacture provide a base for the immobilization of nucleic acid molecules into discrete units. For example, the substrate can be a chip or a membrane. The term “unit” refers to a plurality of nucleic acid molecules containing the same nucleotide sequence variant. Immobilized nucleic acid molecules are typically about 20 nucleotides in length, but can vary from about 15 nucleotides to about 100 nucleotides in length. In practice, a sample of DNA or RNA from a subject can be amplified, hybridized to the article of manufacture, and then hybridization detected. Typically, the amplified product is labeled to facilitate hybridization detection. See, for example, Hacia, J. G. et al., Nature Genetics, 14:441-447 (1996), U.S. Pat. No. 5,770,722, and U.S. Pat. No. 5,733,729.

The invention will be further described in the following examples, which do not limit the scope of the invention described in the claims.

### EXAMPLES

### Example 1

Amplification of Human PRKAG3: Primer sequences specific for the human PRKAG3 gene were derived from a human genomic DNA sequence having GenBank Accession No. AC009974. The primers with their orientations and locations within the gene are listed in Table 1, while the primer combinations used, amplified gene region, PCR annealing temperature, and the expected product sizes are specified in Table 2. Generated products were used for sequence analysis and identification of single nucleotide polymorphisms.

TABLE 1
Primer Sequences

| Primer | Orien- | | |
|---|---|---|---|
| name | tation | Sequence 5′-3′ | Location |
| hRNF12 | Forward | AGG CTC TTG GAA TAG | 5′untran- |
| | | GGG CTC AGG | scribed |
| nRNR13 | Reverse | AGG GAA TTG GGG TCC | intron 2 |
| | | CAG AAA AGT G | |
| hRNF1 | Forward | GAATTGATTTTGATGCATTACTCC | intron 2 |
| hRNR1 | Reverse | AGTGGCGGCTGCAGCACCGT | intron 4 |
| hRNF2.2 | Forward | AGG CAG ATG GGA GGT | Intron 4 |
| | | GCG CAC TGA G | |
| hRNR2.2 | Reverse | ACA GGG ATG GCA TGA | Intron 10 |
| | | GAA ACC CTG C | |
| hRNF4.2 | Forward | TTC TGG TAG TGG CAC | Intron 10 |
| | | CCT GAT GCA A | |
| hRNR3.2 | Reverse | GAC CTG TGA GTC CTT | 3′UTR |
| | | ACA CTT GCA G | |

TABLE 2
PCR Conditions

| | | Annealing | | |
|---|---|---|---|---|
| | Amplified | Temp. | Expected | |
| PCR primers | gene region^{a} | (° C.) | size (bp) | FIG |
| hRNF12 + | 5′untran- | 62 | 873 | 1 |
| hRNR13 | scribed- | | | |
| | intron 2 | | | |
| hRNF1 + | intron 2- | 60-50 | 1042 | 2 |
| hRNR1 | intron 4 | (touch- | | |
| | | down) | | |
| hRNF2.2 + | intron 4- | 60 | 1992 | 3 |
| hRNR2.2 | intron 10 | | | |
| hRNF4.2 + | intron 10- | 60 | 1184 | 4 |
| hRNR3.2 | 3′UTR | | | |

^{a}Location of the start codon of exon 1 in agreement with the human cDNA sequence having GenBank (Accession No. J249977).

PCR reactions for the hRNF12+hRNR13, hRNF2.2+RNR2.2 and hRNF4.2+hRNR3.3 amplicons (see Table 2) were performed in 2 μl reactions including 0.70U AmpliTaq DNA polymerase (Perkin Elmer, Branchburg, N.J., USA), 1×PCR buffer, 1.5 mM MgCl₂, 0.2 mM of each dNTP, 5 pmol of each primer, 5% DMSO, and 20 ng genomic DNA. For these amplicons, thermocycling was carried out using a PTC 100 instrument (MJ Research, Watertown, Mass., USA) and included 40 cycles with annealing at 60-62° C. for 30 s and extension at 72° C. for 1-2 min (see Table 2). The denaturation steps were at 95° C. for 1-2 min in the first two cycles, and at 94° C. for 1 min in the remaining cycles. For the hRNF1+hRNR1 amplicon, the PCR reactions were performed in 20 μl reactions including 0.75U AmpliTaq GOLD DNA polymerase (Perkin Elmer, Branchburg, N.J., USA), 1× GeneAmp GOLD PCR buffer, 1.5 mM MgCl₂, 0.2 mM of each dNTP, 8 pmol of each primer, and 50 ng genomic DNA. For this amplicon, the thermocycling was carried out using a PE9600 (Perkin-Elmer, Foster City, Calif., USA) instrument and included an initial heat activation step at 95° C. for 10 min followed by 45 cycles with denaturation at 95° C. for 30 s, touch-down annealing at 60-50° C. (60° C. followed by one degree decrease per cycle to 50° C. that was then fixed in the remaining cycles) for 30 s and extension at 70° C. for 1 min (see Table 2).

The PCR products were directly sequenced with BigDye terminators and an ABI 377 instrument (Perkin-Elmer, Foster City, Calif., USA). Sequence analysis was carried out using the Sequencher 3.11 software (GENE CODES, Ann Arbor, Mich., USA).

A total of 39 human genomic DNA samples were included in the sequence analysis of the four PCR amplicons described in Table 2. Genomic DNA was prepared from whole blood samples using a standard protocol based on proteinase K treatment of lysed cells, NaCl precipitation for removal of proteins, followed by ethanol precipitation of DNA. Sardinians and Swedes are represented in the sample set that includes a total of 25 diabetes mellitus type 1 (DM1) or diabetes mellitus type II (DM2) patients as well as 14 healthy control individuals. More details about the samples such as sex, age of incidence, and body mass index (BMI) are given in Table 3.

TABLE 3
Patient Information

| | | | | | | Healthy |
|---|---|---|---|---|---|---|
| Sardinian samples | Sex | DM2 | Age | BMI | DM2 sibs | sibs |
| SA912 | M | No | 62 | 29.4 | 2 | 0 |
| SA658 | M | No | 64 | 24.0 | 2 | 1 |
| SA1015 | F | No | 70 | 35.8 | 2 | 0 |
| SA533 | M | No | 60 | 28.7 | 2 | 0 |
| SA656 | M | No | 66 | 32.9 | 1 | 2 |
| SA494 | F | Yes | 42 | 21.4 | 1 | 0 |
| SA548 | M | Yes | 41 | 23.5 | 2 | 0 |
| SA61 | F | Yes | 25 | 26.0 | 1 | 0 |
| SA189 | F | Yes | 58 | 20.5 | 1 | 1 |
| SA1012 | F | Yes | 45 | 21.9 | 3 | 0 |
| | | | | | | Healthy |
| Swedish samples | Sex | DM2 | Age | BMI | DM2 sibs | sibs |
| SW123 | F | No | 58 | 22.9 | — | — |
| SW142 | F | No | 68 | 18.1 | — | — |
| SW166 | F | No | 46 | 24.8 | — | — |
| SW211 | F | No | 70 | 23.5 | — | — |
| SW191 | M | No | 54 | 24.8 | — | — |
| SW582 | M | No | 76 | 28.4 | — | — |
| SW1220 | M | No | 76 | 25.1 | — | — |
| SW1518 | F | No | 72 | 24.1 | — | — |
| SW1906 | F | No | 71 | 25.5 | — | — |
| SW140 | M | Yes | 68 | 29.4 | — | — |
| SW167 | M | Yes | 48 | 30.8 | — | — |
| Swedish samples, | | Susp. | | | | Healthy |
| suspected mody | Sex | MODY | Age | BMI | DM2 sibs | sibs |
| SW1498 | F | Yes | 23 | 25.4 | 2 | 1 |
| SW1507 | F | Yes | 20 | 26.3 | 0 | 1 |
| SW860 | F | Yes | 6 | 13.1 | 3 | 0 |
| SW1464 | M | Yes | 19 | 23.7 | 0 | 2 |
| SW1993 | M | Yes | 32 | 27.8 | 4 | 0 |
| Swedish IDDM | | | | | | Healthy |
| samples | Sex | DM | Age | BMI | DM2 sibs | sibs |
| SW190 | F | DM1 | 51 | 20.1 | — | — |
| X2 | F | DM1 | 22 | 21.6 | — | — |
| X22 | M | DM1 | 31 | 20.9 | — | — |
| X70 | F | DM1 | 35 | 21.0 | — | — |
| X99 | M | DM1 | 21 | 20.8 | — | — |
| X187 | F | DM1 | 22 | 19.8 | — | — |
| X39 | M | DM1 | 35 | 27.5 | — | — |
| X1009 | F | DM1 | 30 | 19.3 | — | — |
| X714 | F | DM1 | 28 | 17.6 | — | — |
| X94 | F | DM1 | 32 | 18.0 | — | — |
| X661 | M | DM1 | 33 | 21.9 | — | — |
| X676 | F | DM1 | 30 | 20.7 | — | — |
| X902 | F | DM1 | 34 | 21.8 | — | — |

### Example 2

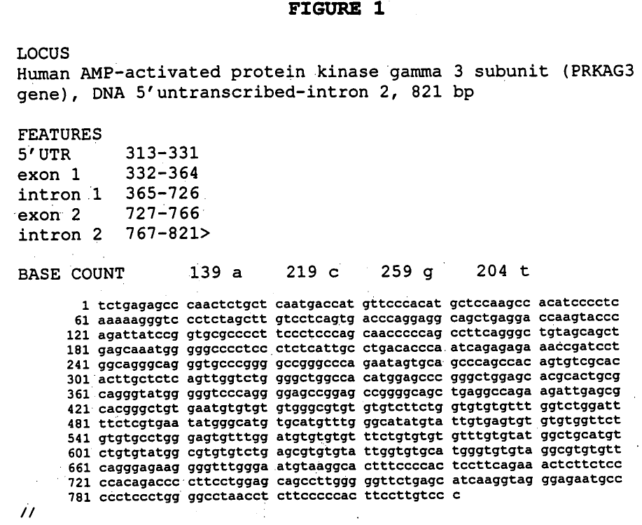

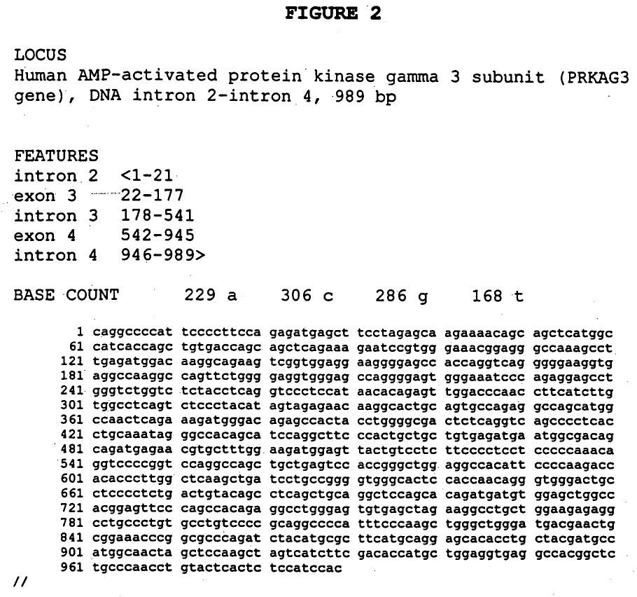

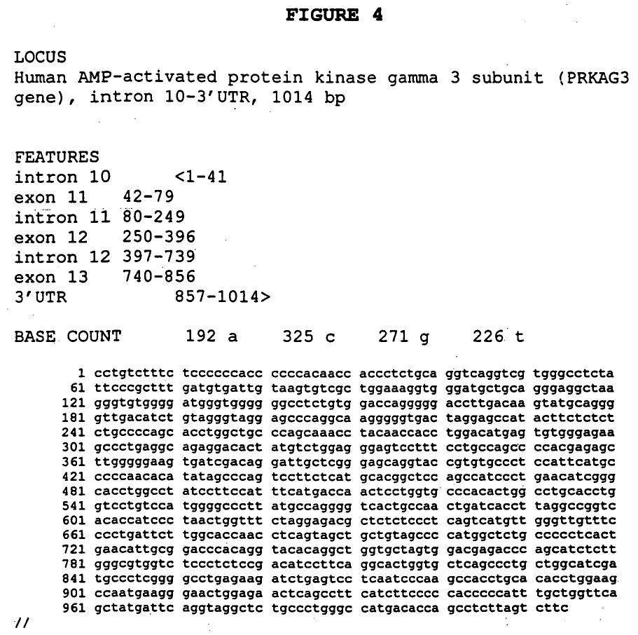

Determination of PRKAG3 specificity and consensus sequences from the four amplicons: PCR products with sizes in agreement with the predicted size (Table 2) were obtained and the desired PRKAG3 gene specificity was confirmed for all four amplicons by sequencing and alignment against the GenBank Accession No. AC009974 sequence. Alignments of sequences from the 39 human samples were used to determine the consensus sequence for each amplicon, and are presented in FIGS. 1-4.

The complete coding PRKAG3 sequence was deduced from the sequences of the four genomic DNA sequences and is shown in FIG. 5. It should be noted that the alignment between this sequence and the cDNA sequence in GenBank (#AJ249977) revealed one single difference that appeared at nucleotide position 1474 in the present sequence. The sequence described herein clearly shows a “G” at this position that is absent at the corresponding position in AJ249977, causing a frameshift and mismatch alignment relative to the amino acid sequence predicted from the present sequence.

The alignments between the 39 human samples revealed four single nucleotide substitutions (single nucleotide polymorphisms, SNP's), which are described in Table 4.

TABLE 4
Single nucleotide polymorphisms in the human PRKAG3 gene

| | Nucleotide | Nucleotide | Predicted amino |
|---|---|---|---|
| Location | position | change | acid change^{a} |
| exon 3 | 230^{a} | C [symbol] →G | P71A |
| exon 4 | 559^{a} | C [symbol] →T | No |
| intron 6 | 642^{b} | G [symbol] →C | — |
| exon 10 | 1037^{a} | C [symbol] →T | R340W |

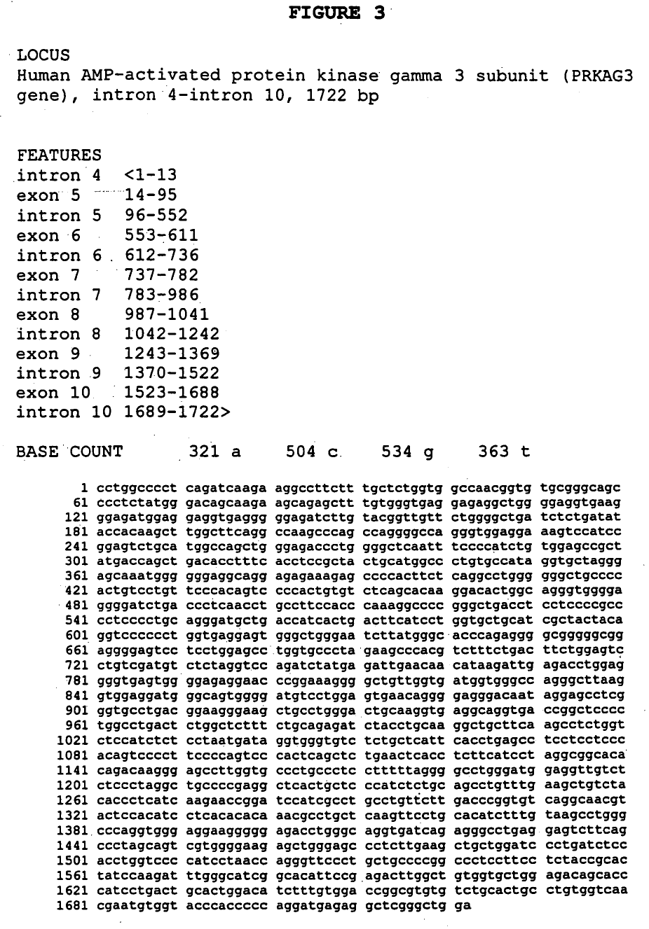

^{a}Position based on the human cDNA sequence in FIG. 5.
^{b}Nucleotide position based on the sequence in FIG. 3.

Two SNP's change the predicted amino acid sequence. The SNP in exon 10 changes the amino acid arginine (R) to tryptophan (W) at amino acid position 340 (R340W based on sequence in FIG. 5 and GenBank Accession No. AJ249977). Substitution of a tryptophan for an arginine is a dramatic change in terms of the electrical charge and chemical characteristics of the amino acid, which indicates a possible effect on protein function. Moreover, the data indicate that the R340W variant was over-represented among diabetes patients. Four patients with diabetes (two Type I, one Type II, and one with Type I or Type II) and one control were found to have this variant.

A variety of available molecular genetic techniques for SNP detection can be used to screen the SNPs in Table 4, as described above. PCR primers hRNF9 (5′ GCT GGA TCC CG ATC TCC ACC TG, forward, intron9) and hRNR10(5′CGT TGA CCA CAG GCA GTG CAG AC, reverse, exon10) were designed from the FIG. 3 sequence and used for PCR amplification of a 200 bp fragment containing the SNP in exon 10. The PCR reactions were performed in 10 μl reactions including 0.35 U AmpliTaq DNA polymerase (Perkin Elmer, Branchburg, N.J., USA), 1×PCR buffer, 1.5 mM MgCl₂, 0.2 mM of each dNTP, 2.5 pmol of each primer, 5% DMSO, and 10 ng genomic DNA. Thermocycling was carried out using a PTC 100 instrument (MJ Research, Watertown, Mass., USA). The thermocycling included 40 cycles with annealing at 61° C. for 30 s and extension at 72° C. for 30 s. The denaturation step was at 95° C. for 2 min in the first cycles, and at 94° C. for 1 min in the remaining cycles. Four μl of each PCR product were digested in 10 μl with 2.4 U MspI (New England Biolabs, Frankfurt am Main, Germany) containing the buffer recommended by the manufacturer. The digestions were analyzed by 6% Nusieve/Seakem 3:1 agarose (FMC Bioproducts, Rockland, Me., USA) gel electrophoresis and visualization of the DNA fragments by ethidium bromide staining and WV illumination. Digestion with Msp I generated allelic fragments of 169 bp (allele I), 114 and 55 bp (allele 2) as well as the monomorphic fragment 31 bp. Homozygous 2/2 genotypes and heterozygous 1/2 genotypes were observed.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An isolated nucleic acid comprising a human PRKAG3 sequence, wherein said human PRKAG3 sequence comprises a nucleotide sequence variant and nucleotides flanking said sequence variant, and wherein said isolated nucleic acid is at least 15 base pairs in length.

2. The nucleic acid of claim 1, wherein said nucleotide sequence variant is associated with a metabolic disease.

3. The nucleic acid of claim 2, wherein said metabolic disease is diabetes or obesity.

4. The nucleic acid of claim 1, wherein said nucleotide sequence variant is in an exon.

5. The nucleic acid of claim 4, wherein said exon is selected from the group consisting of exon 3, exon 4, and exon 10.

6. The nucleic acid of claim 4, wherein said exon 3 variant comprises a substitution of a guanine for a cytosine at nucleotide 230.

7. The nucleic acid of claim 4, wherein said exon 4 variant comprises a substitution of a thymine for a cytosine at nucleotide 550.

8. The nucleic acid of claim 4, wherein said exon 10 variant comprises a substitution of a thymine for a cytosine at nucleotide 1037.

9. The nucleic acid of claim 1, wherein said nucleotide sequence variant is in an intron.

10. The nucleic acid of claim 9, wherein said nucleotide sequence variant is in intron 6.

11. The nucleic acid of claim 1, wherein said PRKAG3 nucleic acid sequence encodes an AMP-activated protein kinase γ3 subunit polypeptide, said polypeptide comprising an amino acid sequence variant.

12. The nucleic acid of claim 11, wherein said amino acid sequence variant comprises substitution of an alanine residue for a proline residue at amino acid 71.

13. The nucleic acid of claim 11, wherein said amino acid sequence variant comprises substitution of a tryptophan residue for an arginine residue at amino acid 340.

14. A method for determining a risk estimate of a metabolic disease in a subject, said method comprising detecting the presence or absence of a PRKAG3 nucleotide sequence variant in said subject, and determining said risk estimate based, at least in part, on presence or absence of said variant in said subject.

15. The method of claim 14, wherein said metabolic disease is diabetes or obesity.

16. A method for detecting a PRKAG3 polypeptide variant in a subject, said method comprising providing a biological sample from said subject, contacting said biological sample with an antibody having specific binding affinity for said PRKAG3 polypeptide variant, and detecting the presence or absence of said PRKAG3 polypeptide variant in said biological sample.

17. An article of manufacture comprising a substrate and an array of different nucleic acids immobilized on said substrate, wherein at least one of said different nucleic acids is a PRKAG3 nucleic acid, and wherein said PRKAG3 nucleic acid comprises a PRKAG3 nucleotide sequence variant and nucleotides flanking said sequence variant.

18. The article of manufacture of claim 17, wherein said array comprises multiple PRKAG3 nucleic acids, wherein each of said PRKAG3 nucleic acids comprises a different PRKAG3 nucleotide sequence variant and nucleotides flanking said sequence variant.


SEQUENCE LISTING (16 sequences)

SEQ ID NO: 1 - DNA, 821 bp, Homo sapiens
tctgagagcccaactctgctcaatgaccatgttcccacatgctccaagccacatcccctcaaaaagggtccctctagcttgtcctcagtgacccaggaggcagctgaggaccaagtacccagattatccggtgcgccccttccctcccagcaacccccagccttcagggctgtagcagctgagcaaatgggggcccctccctctcattgcctgacacccaatcagagagaaaccgatcctggcagggcagggtgcccggggccgggcccagaatagtgcagcccagccacagtgtcgcacacttgctctcagttggtctggggctggccacatggagcccgggctggagcacgcactgcgcagggtatgggggtcccaggggagccggagccggggcagctgaggccagaagattgagcgcacgggctgtgaatgtgtgtgtgggcgtgtgtgtcttctggtgtgtgtttggtctggattttctcgtgaatatgggcatgtgcatgtttgggcatatgtattgtgagtgtgtgtggttctgtgtgcctgggagtgtttggatgtgtgtgtttctgtgtgtgtttgtgtatggctgcatgtctgtgtatggcgtgtgtctgagcgtgtgtattggtgtgcatgggtgtgtaggcgtgtgttcagggagaaggggtttgggaatgtaaggcactttccccactccttcagaaactcttctccccacagaccccttcctggagcagccttgggggttctgagcatcaaggtagggagaatgccccctccctggggcctaacctcttcccccacttccttgtccc

SEQ ID NO: 2 - DNA, 989 bp, Homo sapiens
caggccccattccccttccagagatgagcttcctagagcaagaaaacagcagctcatggccatcaccagctgtgaccagcagctcagaaagaatccgtgggaaacggagggccaaagccttgagatggacaaggcagaagtcggtggaggaaggggagccaccaggtcagggggaaggtgaggccaaggccagttctggggaggtgggagccaggggagtgggaaatcccagaggagcctgggtctggtctctacctcaggtccctccataacacagagttggacccaaccttcatcttgtggcctcagtctccctacatagtagagaacaaggcactgcagtgccagaggccagcatggccaactcagaaagatgggacagagccactacctggggcgactctcaggtcagcccctcacctgcaaatagggccacagcatccaggcttcccactgctgctgtgagatgaatggcgacagcagatgagaacgtgctttggaagatggagttactgtcctcttcccctcctcccccaaacaggtccccggtccaggccagctgctgagtccaccgggctggaggccacattccccaagaccacacccttggctcaagctgatcctgccggggtgggcactccaccaacagggtgggactgcctcccctctgactgtacagcctcagctgcaggctccagcacagatgatgtggagctggccacggagttcccagccacagaggcctgggagtgtgagctagaaggcctgctggaagagaggcctgccctgtgcctgtccccgcaggccccatttcccaagctgggctgggatgacgaactgcggaaacccggcgcccagatctacatgcgcttcatgcaggagcacacctgctacgatgccatggcaactagctccaagctagtcatcttcgacaccatgctggaggtgaggccacggctctgcccaacctgtactcactctccatccac

SEQ ID NO: 3 - DNA, 1722 bp, Homo sapiens
cctggcccctcagatcaagaaggccttctttgctctggtggccaacggtgtgcgggcagcccctctatgggacagcaagaagcagagctttgtgggtgaggagaggctggggaggtgaagggagatggaggaggtgagggggagatcttgtacggttgttctggggctgatctctgatataccacaagcttggcttcaggccaagcccagccaggggccagggtggaggaaagtccatccggagtctgcatggccagctgggagaccctggggctcaatttccccatctgtggagccgctatgaccagctgacacctttcacctccgctactgcatggccctgtgccataggtgctagggagcaaatggggggaggcaggagagaaagagccccacttctcaggcctggggggctgccccactgtcctgttcccacagtccccactgtgtctcagcacaaggacactggcagggtggggaggggatctgaccctcaacctgccttccacccaaaggccccgggctgacctcctccccgcccctcccctgcagggatgctgaccatcactgacttcatcctggtgctgcatcgctactacaggtcccccctggtgaggagtgggctgggaatcttatgggcacccagaggggcgggggcggaggggagtcctcctggagcctggtgccctagaagcccacgtctttctgacttctggagtcctgtcgatgtctctaggtccagatctatgagattgaacaacataagattgagacctggaggggtgagtggggagaggaacccggaaaggggctgttggtgatggtgggccagggcttaaggtggaggatgggcagtggggatgtcctggagtgaacaggggagggacaataggagcctcgggtgcctgacggaagggaagctgcctgggactgcaaggtgaggcaggtgaccggctcccctggcctgactctggctctttctgcagagatctacctgcaaggctgcttcaagcctctggtctccatctctcctaatgataggtgggtgtctctgctcattcacctgagcctcctcctcccacagtccccttccccagtcccactcagctctgaactcacctcttcatcctaggcggcacacagacaagggagccttggtgccctgccctcctttttaggggcctgggatggaggttgtctctccctaggctgccccgaggctcactgctcccatctctgcagcctgtttgaagctgtctacaccctcatcaagaaccggatccatcgcctgcctgttcttgacccggtgtcaggcaacgtactccacatcctcacacacaaacgcctgctcaagttcctgcacatctttgtaagcctgggcccaggtgggaggaagggggagacctgggcaggtgatcagagggcctgaggagtcttcagccctagcagtcgtggggaagagctgggagccctcttgaagctgctggatccctgatctccacctggtccccatcctaaccagggttccctgctgccccggccctccttcctctaccgcactatccaagatttgggcatcggcacattccgagacttggctgtggtgctggagacagcacccatcctgactgcactggacatctttgtggaccggcgtgtgtctgcactgcctgtggtcaacgaatgtggtacccacccccaggatgagaggctcgggctgga

SEQ ID NO: 4 - DNA, 1014 bp, Homo sapiens
cctgtctttctccccccaccccccacaaccaccctctgcaggtcaggtcgtgggcctctattcccgctttgatgtgattgtaagtgtcgctggaaaggtgggatgctgcagggaggctaagggtgtggggatgggtggggggcctctgtggaccagggggaccttgacaagtatgcaggggttgacatctgtagggtaggagcccaggcaagggggtgactaggagccatacttctctctctgccccagcacctggctgcccagcaaacctacaaccacctggacatgagtgtgggagaagccctgaggcagaggacactatgtctggagggagtcctttcctgccagccccacgagagcttgggggaagtgatcgacaggattgctcgggagcaggtaccgtgtgccctccattcatgcccccaacacatatagcccagtccttctcatgcacggctccagccatccctgaacatcgggcacctggcctatccttccatttcatgaccaactcctggtgcccacactggcctgcacctggtcctgtccatggggcccttatgccaggggtcactgccaactgatcaccttaggccggtcacaccatccctaactggtttctaggagacgctctctccctcagtcatgttgggttgtttcccctgattcttggcaccaacctcagtagctgctgtagccccatggctctgccccctcactgaacattgcggacccacaggtacacaggctggtgctagtggacgagacccagcatctcttgggcgtggtctccctctccgacatccttcaggcactggtgctcagccctgctggcatcgatgccctcggggcctgagaagatctgagtcctcaatcccaagccacctgcacacctggaagccaatgaagggaactggagaactcagccttcatcttcccccacccccatttgctggttcagctatgattcaggtaggctctgccctgggccatgacaccagcctcttagtcttc

SEQ ID NO: 5 - DNA, 1647 bp, Homo sapiens
  CDS (20)...(1486)
ttggtctggggctggccacatggagcccgggctggagcacgcactgcgcaggaccccttcctggagcagccttgggggttctgagcatcaagagatgagcttcctagagcaagaaaacagcagctcatggccatcaccagctgtgaccagcagctcagaaagaatccgtgggaaacggagggccaaagccttgagatggacaaggcagaagtcggtggaggaaggggagccaccaggtcagggggaaggtccccggtccaggccagctgctgagtccaccgggctggaggccacattccccaagaccacacccttggctcaagctgatcctgccggggtgggcactccaccaacagggtgggactgcctcccctctgactgtacagcctcagctgcaggctccagcacagatgatgtggagctggccacggagttcccagccacagaggcctgggagtgtgagctagaaggcctgctggaagagaggcctgccctgtgcctgtccccgcaggccccatttcccaagctgggctgggatgacgaactgcggaaacccggcgcccagatctacatgcgcttcatgcaggagcacacctgctacgatgccatggcaactagctccaagctagtcatcttcgacaccatgctggagatcaagaaggccttctttgctctggtggccaacggtgtgcgggcagcccctctatgggacagcaagaagcagagctttgtggggatgctgaccatcactgacttcatcctggtgctgcatcgctactacaggtcccccctggtccagatctatgagattgaacaacataagattgagacctggagggagatctacctgcaaggctgcttcaagcctctggtctccatctctcctaatgatagcctgtttgaagctgtctacaccctcatcaagaaccggatccatcgcctgcctgttcttgacccggtgtcaggcaacgtactccacatcctcacacacaaacgcctgctcaagttcctgcacatctttggttccctgctgccccggccctccttcctctaccgcactatccaagatttgggcatcggcacattccgagacttggctgtggtgctggagacagcacccatcctgactgcactggacatctttgtggaccggcgtgtgtctgcactgcctgtggtcaacgaatgtggtcaggtcgtgggcctctattcccgctttgatgtgattcacctggctgcccagcaaacctacaaccacctggacatgagtgtgggagaagccctgaggcagaggacactatgtctggagggagtcctttcctgccagccccacgagagcttgggggaagtgatcgacaggattgctcgggagcaggtacacaggctggtgctagtggacgagacccagcatctcttgggcgtggtctccctctccgacatccttcaggcactggtgctcagccctgctggcatcgatgccctcggggcctgagaagatctgagtcctcaatcccaagccacctgcacacctggaagccaatgaagggaactggagaactcagccttcatcttcccccacccccatttgctggttcagctatgattcaggtaggctctgccctgggccatgacaccagcctcttagtcttc

SEQ ID NO: 6 - protein, 489 aa, Homo sapiens
MEPGLEHALRRTPSWSSLGGSEHQEMSFLEQENSSSWPSPAVTSSSERIRGKRRAKALRWTRQKSVEEGEPPGQGEGPRSRPAAESTGLEATFPKTTPLAQADPAGVGTPPTGWDCLPSDCTASAAGSSTDDVELATEFPATEAWECELEGLLEERPALCLSPQAPFPKLGWDDELRKPGAQIYMRFMQEHTCYDAMATSSKLVIFDTMLEIKKAFFALVANGVRAAPLWDSKKQSFVGMLTITDFILVLHRYYRSPLVQIYEIEQHKIETWREIYLQGCFKPLVSISPNDSLFEAVYTLIKNRIHRLPVLDPVSGNVLHILTHKRLLKFLHIFGSLLPRPSFLYRTIQDLGIGTFRDLAVVLETAPILTALDIFVDRRVSALPVVNECGQVVGLYSRFDVIHLAAQQTYNHLDMSVGEALRQRTLCLEGVLSCQPHESLGEVIDRIAREQVHRLVLVDETQHLLGVVSLSDILQALVLSPAGIDALGA

SEQ ID NO: 7 - DNA, 24 bp, Artificial Sequence
  Synthetically generated primer
aggctcttggaataggggctcagg

SEQ ID NO: 8 - DNA, 25 bp, Artificial Sequence
  Synthetically generated primer
agggaattggggtcccagaaaagtg

SEQ ID NO: 9 - DNA, 24 bp, Artificial Sequence
  Synthetically generated primer
gaattgattttgatgcattactcc

SEQ ID NO: 10 - DNA, 20 bp, Artificial Sequence
  Synthetically generated primer
agtggcggctgcagcaccgt

SEQ ID NO: 11 - DNA, 25 bp, Artificial Sequence
  Synthetically generated primer
aggcagatgggaggtgcgcactgag

SEQ ID NO: 12 - DNA, 25 bp, Artificial Sequence
  Synthetically generated primer
acagggatggcatgagaaaccctgc

SEQ ID NO: 13 - DNA, 25 bp, Artificial Sequence
  Synthetically generated primer
ttctggtagtggcaccctgatgcaa

SEQ ID NO: 14 - DNA, 25 bp, Artificial Sequence
  Synthetically generated primer
gacctgtgagtccttacacttgcag

SEQ ID NO: 15 - DNA, 22 bp, Artificial Sequence
  Primer
gctggatcccgatctccacctg

SEQ ID NO: 16 - DNA, 23 bp, Artificial Sequence
  Primer
cgttgaccacaggcagtgcagac